(19) United States
(12) Patent Application Publication
(10) Pub. No.: US 2024/0130649 A1
(43) Pub. Date: Apr. 25, 2024

(54) Factory Calibration of a Sensor

(71) Applicant: Zense-Life Inc., Carlsbad, CA (US)

(72) Inventors: Robert James Boock, Carlsbad, CA (US); Huashi Zhang, San Juan Capistrano, CA (US); Nelson Chun Man To, Toronto (CA); Sai Kit Wu, Irvine, CA (US); Michael Wheelock, San Clemente, CA (US); Sarah Esparza, Carlsbad, CA (US); Qinyi Yan, San Diego, CA (US)

(73) Assignee: Zense-Life Inc., Carlsbad, CA (US)

(21) Appl. No.: 18/489,311

(22) Filed: Oct. 17, 2023

Related U.S. Application Data
(60) Provisional application No. 63/380,392, filed on Oct. 21, 2022.

(51) Int. Cl. A61B 5/1495; A61B 5/145; A61B 5/1486

(52) U.S. Cl. CPC A61B 5/1495; A61B 5/14532; A61B 5/14865; A61B 2560/0223

(57) ABSTRACT

A method for factory calibration of a sensor of a CGM system is disclosed. A processor receives an enzyme membrane thickness after each dip of a working wire according to first parameters. The processor receives a glucose limiting membrane thickness after each dip of the working wire according to second parameters. The processor determines a working wire diameter, and includes the membrane thicknesses. The processor in communication with the CGM system, automatically generates a correlation between the first and the second parameters to at least one of a factory sensitivity and a drift profile. The drift profile predicts a sensitivity of the sensor over time. The processor associates the at least one of the factory sensitivity or the drift profile with the sensor. The sensor outputs a glucose reading during in-vivo use based on the at least one of the factory sensitivity or the drift profile.

### RELATED APPLICATIONS

This application claims priority to U.S. Provisional Patent Application No. 63/380,392 filed on Oct. 21, 2022, and entitled “Factory Calibration of a Sensor,” which is hereby incorporated by reference in full.

### BACKGROUND

Medical patients often have diseases or conditions that require the measurement and reporting of biological conditions. For example, if a patient has diabetes, it is important that the patient have an accurate understanding of the level of glucose in their blood. Traditionally, diabetes patients have monitored their glucose levels by sticking their finger with a small lancet, allowing a drop of blood to form, and then dipping a test strip into the blood. The test strip is positioned in a handheld monitor that performs an analysis on the blood and visually reports the measured glucose level to the patient. Based upon this reported level, the patient makes important decisions on what food to consume, or how much insulin to inject into their blood. Although it would be advantageous for the patient to check glucose levels many times throughout the day, many patients fail to adequately monitor their glucose levels due to the pain and inconvenience. As a result, the patient may eat improperly or inject either too much or too little insulin. Either way, the patient has a reduced quality of life and increased chance of doing permanent damage to their health and body. Diabetes is a devastating disease that if not properly controlled can lead to terrible physiological conditions such as kidney failure, skin ulcers, or bleeding in the eyes, and eventually blindness, and pain and the eventual amputation of limbs.

Regular and accurate monitoring of glucose levels is critical for diabetes patients. To facilitate such monitoring, continuous glucose monitoring (CGM) sensors are a type of device in which glucose is automatically measured from fluid sampled in an area just under the skin multiple times a day. CGM devices typically involve a small housing in which the electronics are located and which is adhered to the patient's skin to be worn for a period of time. A small needle within the device delivers the subcutaneous sensor which is often electrochemical. In this way, a patient may install a CGM on their body, and the CGM will provide automated and accurate glucose monitoring for many days without any action required from the patient or a caregiver. It will be understood that depending upon the patient's needs, continuous glucose monitoring may be performed at different intervals. For example, some continuous glucose monitors may be set or programmed to take multiple readings per minute, whereas in other cases the continuous glucose monitor can be programmed or set to take readings every hour or so. It will be understood that a continuous glucose monitor may sense and report readings at different intervals.

Continuous glucose monitoring is a complicated process, and it is known that glucose levels in the blood can significantly rise/increase or lower/decrease quickly, due to several causes. Accordingly, a single glucose measurement provides only a snapshot of the instantaneous level of glucose in a patient's body. Such a single measurement provides little information about how the patient's use of glucose is changing over time, or how the patient reacts to specific dosages of insulin. Even a patient that is adhering to a strict schedule of strip testing will likely be making incorrect decisions as to diet, exercise, and insulin injection. Of course, this is exacerbated by a patient that is less consistent on performing their strip testing. To give the patient a more complete understanding of their diabetic condition and to get a better therapeutic result, some diabetic patients are now using continuous glucose monitoring.

Electrochemical glucose sensors operate by using electrodes which typically detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is biased. The reference electrodes essentially complete the electron flow in the electrochemical circuit. Three-electrode (or three-pole) designs have a working electrode, a reference electrode and a counter electrode. The counter electrode replenishes ionic loss at the reference electrode and is part of an ionic circuit.

The working wire is then associated with a reference electrode, and in some cases one or more counter electrodes, which form the CGM sensor. In operation, the CGM sensor is coupled to and cooperates with electronics in a small housing in which, for example, a processor, memory, a wireless radio, and a power supply are located. The CGM sensor typically has a disposable applicator device that uses a small introducer needle to deliver the CGM sensor subcutaneously into the patient. Once the CGM sensor is in place, the applicator is discarded, and the electronics housing is attached to the sensor. Although the electronics housing is reusable and may be used for extended periods, the CGM sensor and applicator need to be replaced quite often, usually every few days.

A significant deficiency in known CGM sensors is that they exhibit substantial variability patient to patient, and even have sensitivity variability for a given patient over time. More particularly, the CGM sensors have variations in sensitivity to blood glucose concentrations, and so must be locally calibrated by each patient prior to use, and then re-calibrated over time for a particular user. Unfortunately, the local calibration processes require the patient pricking their finger and obtaining a blood glucose reading using a standard strip monitor. Not only is local calibration inconvenient, time consuming, and prone to error, it is painful such that a patient may delay or avoid local calibration, thereby defeating any possible benefit from the CGM system.

It is known in the art that after the sensor of the CGM is implanted, it is calibrated by the user, and the sensor provides sensor data to the electronics. The electronics convert the sensor data so that estimated glucose levels in the blood can be continuously reported to the user. As the electronics continue to receive sensor data, the sensor may be locally recalibrated by the user to account for possible changes in sensor sensitivity. Sensor sensitivity is a measure of the reaction of glucose oxidase which from that reaction produces hydrogen peroxide that is measured directly on the working electrode surface as free electrons that create current. Over time, sensitivity may change due to a variety of factors which can affect an accurate measurement so it is known in the art that it is necessary to calibrate the sensor daily or several times a week.

### SUMMARY

In some embodiments, a method for factory calibration of a sensor of a continuous glucose monitoring (CGM) system is disclosed. A processor receives enzyme membrane data including an enzyme membrane thickness after each dip of a working wire in a first dip solution according to first parameters for forming an enzyme membrane on the working wire. The processor receives glucose limiting membrane data including a glucose limiting membrane thickness after each dip of the working wire in a second dip solution according to second parameters for forming a glucose limiting membrane on the working wire. The processor determines a working wire diameter after the working wire is formed. The working wire diameter includes the enzyme membrane thickness and the glucose limiting membrane thickness. The formed working wire includes an interference membrane, the enzyme membrane and the glucose limiting membrane. The processor in communication with the CGM system, automatically generates a correlation between the first parameters and the second parameters to at least one of a factory sensitivity and a drift profile of the sensor. The drift profile predicts a sensitivity of the sensor over time. The processor associates the at least one of the factory sensitivity or the drift profile with the sensor. The sensor outputs a glucose reading during in-vivo use based on the at least one of the factory sensitivity or the drift profile.

In some embodiments, a method for factory calibration of a sensor of a continuous glucose monitoring (CGM) system is disclosed. A working wire is dipped in a first coating solution according to first parameters to form an enzyme membrane on the working wire. Enzyme membrane data including an enzyme membrane thickness is measured. The working wire is dipped in a second coating solution according to second parameters to form a glucose limiting membrane on the wire. Glucose limiting membrane data including a glucose limiting membrane thickness is measured. A processor determines a working wire diameter after the working wire is formed. The working wire diameter includes the enzyme membrane thickness and the glucose limiting membrane thickness. The formed working wire includes an interference membrane, the enzyme membrane and the glucose limiting membrane. The processor in communication with the CGM system, automatically generates a correlation between the first parameters and the second parameters to at least one of a factory sensitivity, and a drift profile of the sensor. The drift profile predicts a sensitivity of the sensor over time. The processor associates the at least one of the factory sensitivity or the drift profile with the sensor. The sensor outputs a glucose reading during in-vivo use based on the at least one of the factory sensitivity or the drift profile.

### BRIEF DESCRIPTION OF THE DRAWINGS

FIG. 1 is a cross-sectional view of a working wire, in accordance with some embodiments.

FIG. 2 is a process flow block diagram for manufacturing a working wire and a reference wire, in accordance with some embodiments.

FIG. 3 depicts an example of data sampling after skiving and singulation during manufacturing of a sensor wire, in accordance with some embodiments.

FIG. 4 depicts an example of data sampling after electropolymerization during manufacturing of a sensor wire, in accordance with some embodiments.

FIG. 5 is an isometric view of a dipping station having a fixture and a tub, in accordance with some embodiments.

FIG. 6 depicts an example of data sampling after forming the enzyme membrane, in accordance with some embodiments.

FIGS. 7A-7C depict examples of data samplings after forming the glucose limiting membrane on day one, day two and day three, in accordance with some embodiments.

FIG. 7D shows a sensitivity graph of the electrical response for a prior art CGM

sensor.

FIG. 8A depicts linearity statistics for sensors for a calibration check, in accordance with some embodiments.

FIG. 8B depicts sensitivity statistics for sensors during a calibration check, in accordance with some embodiments.

FIG. 8C is a graph of baseline of the sensor and sensitivity of the sensor, in accordance with some embodiments.

FIG. 8D is a graph of the log of baseline of the sensor and the log of sensitivity of the sensor from the data in FIG. 8C, in accordance with some embodiments.

FIG. 8E is a graph of in-vivo baseline and in-vivo sensitivity, in accordance with some embodiments.

FIG. 8F is a graph of in-vivo sensitivity drift and in-vivo sensitivity, in accordance with some embodiments.

FIGS. 9A and 9B are flowcharts for factory calibrations of a sensor of a continuous glucose monitoring (CGM) system, in accordance with some embodiments.

FIG. 10 is a schematic of a CGM system in-use by a patient, in accordance with some embodiments.

FIGS. 11A-11C are tables detailing process improvements, in accordance with some embodiments.

FIG. 12 illustrates an automated factory software control process, in accordance with some embodiments.

FIG. 13 is a simplified schematic diagram showing an example computer system for use in the computing platform, in accordance with some embodiments.

### DETAILED DESCRIPTION

The present disclosure relates to performing a factory calibration of continuous glucose monitoring (CGM) systems. Embodiments disclose that after undergoing calibration at the factory, there is no subsequent need for recalibration. For example, once the sensor has been calibrated in the factory, it does not need to be calibrated again. By eliminating the necessity of user-performed local calibration, there is no longer a requirement for the frequent finger-prick method to obtain blood glucose measurements for in-use calibration, thus allowing it to be completely avoided.

Systems and processes for manufacturing sensor wires for a continuous biological sensor are described. The continuous biological sensor may be, for example, a continuous glucose monitor. In some embodiments, the sensor may be a two-electrode design having a working electrode and a reference electrode, where the reference electrode provides a reference against which the working electrode is compared. The working wire includes an enzyme membrane to detect the level of glucose in a patient's blood. In other embodiments, the biological sensor can be a metabolic sensor for measuring other metabolic characteristics such as ketones, lactates or fatty acids. The sensor uses a working wire that is the electrode for the sensor, and has several concentrically formed membrane layers.

In some embodiments, an automated system measures dimensions of working wires while the working wires undergo a dipping process to form the membranes (also referred to as layers), then utilizes these measurements to make real-time adjustments to dipping parameters. The measurement system takes multiple measurements along a length of the working wire while measuring multiple wires that are mounted in a carrier. By providing thorough monitoring of coating thicknesses in an in-line manner while the layers are being built, more efficient and accurate dip coating of working wires is achieved. The in-line manner involves taking measurements during a dipping process, after each dip for creating a layer. In contrast, conventional methods typically take measurements after all the layers on the wire have been applied or formed. The present methods adjust dipping parameters based on the measured thicknesses after each dip, and on other factors that are monitored such as the temperature or viscosity of the dip solution (also referred to as coating solution). In some embodiments, environmental factors can also be analyzed along with the coated wire measurements to adjust dipping parameters. The systems and methods may optimize the manufacturing process, such as by reducing the number of dips required to achieve a desired coating thickness within a target window.

In some embodiments, the measurement system provides the thickness of each membrane of the working wire of the sensor such as the enzyme membrane and the glucose limiting membrane according to dipping parameters and environmental factors. The dipping parameters may include at least one of dip solution viscosity, dip solution temperature, immersion speed, dwell time, withdrawal speed, and airflow. The environmental factors may include air temperature, airflow velocity and relative humidity of the airflow. The dipping parameters and environmental factors are monitored and tracked during the formation of the membranes.

Embodiments herein create a factory calibration by using a working wire diameter and sensor sensitivity of the sensor after the interference membrane, enzyme membrane and glucose limiting membrane are formed. The working wire diameter includes the enzyme membrane thickness and the glucose limiting membrane thickness. A correlation between the first parameters for the enzyme membrane and second parameters for the glucose limiting membrane to at least one of a factory sensitivity and a drift profile of the sensor is determined. The factory sensitivity or the drift profile is associated with the sensor. Accordingly, the sensor while in use outputs a glucose reading based on the at least one of the factory sensitivity or the drift profile. By implementing this approach, the need for users to perform frequent local calibrations while the sensor is in use is eliminated, rendering the frequent finger-prick method for obtaining in-use calibration blood glucose measurements unnecessary. Consequently, ease-of-use is greatly improved for the patient while maintaining or improving accuracy of the sensor readings.

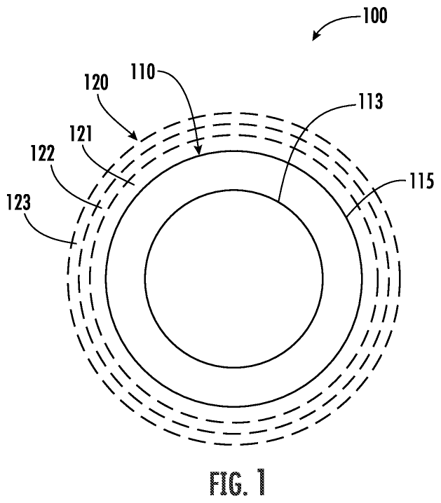

Referring to FIG. 1, a cross-sectional view of a working wire 100 is illustrated in accordance with some embodiments. In this example, the working wire 100 is an elongated wire having a circular cross-section. It will be understood that other cross-sections may be used, such as square, rectangular, triangular, or other geometric shapes. Furthermore, the working wire 100 may take other forms, such as a plate or ribbon. The working wire is used as a working electrode of a continuous biological sensor, such as a working electrode of a continuous glucose monitor.

In the illustrated example, the working wire 100 has a substrate 110 onto which biological membranes 120 may be disposed. The types of biological membranes that may be used are well-known and will not be described in detail herein. In one example as illustrated, the biological membranes 120 include an interference membrane 121 (which may also be referred to as an interference layer) on the substrate 110, an enzyme membrane 122 (i.e., enzyme layer) on the interference membrane 121, and a glucose limiting membrane 123 (i.e., glucose limiting layer) on the enzyme membrane 122. In some embodiments, a protective or outer coating may be optionally applied over the glucose limiting membrane 123. Although the working wire 100 is illustrated as having three membranes 120, it will be understood that the membranes 120 may be more or fewer in number.

The substrate 110 may be comprised of a core 113 with an outer layer 115. In the example of FIG. 1, the core 113 is an elongated wire that is dense, ductile, very hard, easily fabricated, highly conductive of heat and electricity, and may also be resistant to corrosion. Example materials for core 113 include tantalum, carbon, or cobalt-chromium (Co—Cr) alloys. The core 113 may have the outer layer 115, such as of platinum, deposited or applied using an electroplating process. It will be understood that other processes may be used for applying the outer layer 115 to the core 113. For a glucose monitor, the platinum outer layer facilitates a reaction where the hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which in turn is proportional to the number of glucose molecules oxidized. A measurement of the electrical current on the platinum wire can thereby be associated with a particular level of glucose in the patient's blood or interstitial fluid (ISF).

The core 113, outer layer 115, interference membrane 121, and enzyme membrane 122 form key aspects of working wire 100. Additional layers or membranes can be incorporated as needed, depending on the specific biological substance being tested and the unique requirements of the application. In some cases, the core 113 may have an inner core portion (not shown). For example, if the substrate (core 113) is made from tantalum, an inner core of titanium or titanium alloy may be included to provide additional strength and straightness.

In some cases, one or more membranes (i.e., layers) may be provided over the enzyme membrane 122. For example, a glucose limiting membrane 123 may be layered on top of the enzyme membrane 122. This glucose limiting membrane 123 may limit the number of glucose molecules that can pass through the glucose limiting membrane 123 and into the enzyme membrane 122. The glucose limiting membrane 123 can be configured as described in U.S. Pat. No. 11,576,595, entitled “Enhanced Sensor for a Continuous Biological Monitor,” which is owned by the assignee of the present disclosure and is incorporated herein by reference as if set forth in its entirety. In some cases, the addition of the glucose limiting membrane 123 has been shown to enable better performance of the overall working wire 100.

An interference membrane 121 is applied over the outer layer 115. The interference membrane 121 may be disposed between the enzyme membrane 122 and the outer layer 115. This interference membrane 121 is constructed to fully wrap the outer layer 115, thereby protecting the outer layer 115 from further oxidation effects. The interference membrane 121 is also constructed to substantially restrict the passage of larger molecules, such as acetaminophen, to reduce contaminants that can reach the platinum and skew results. Further, the interference membrane 121 may pass a controlled level of hydrogen peroxide (H₂O₂) from the enzyme membrane 122 to the platinum outer layer 115. Compositions for the interference membrane 121 and enzyme membrane 122 may be as described in U.S. patent application Ser. No. 17/449,562, entitled “Working Wire for a Continuous Biological Sensor with an Immobilization Network,” and U.S. patent application Ser. No. 17/449,380, entitled “In-Vivo Glucose Specific Sensor,” which are owned by the assignee of the present disclosure and incorporated herein by reference as if set forth in their entirety.

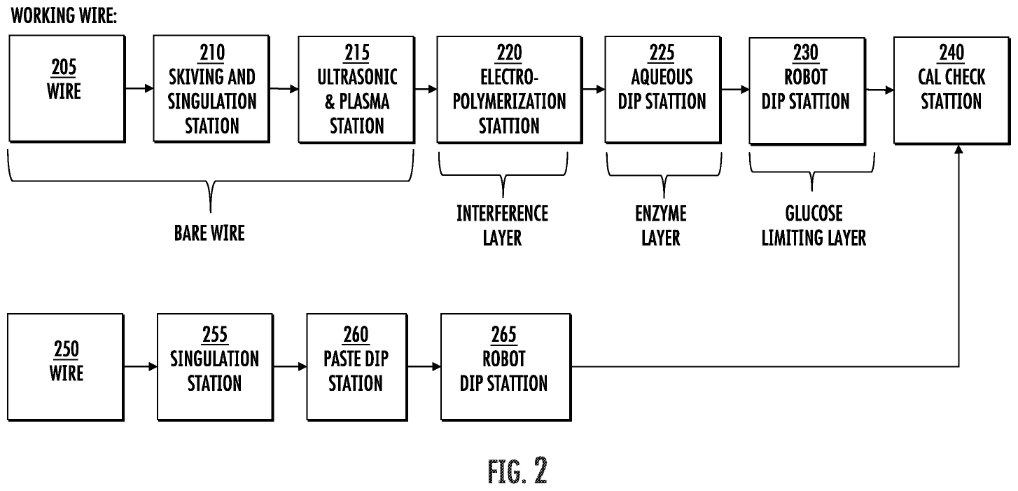

FIG. 2 is a process flow block diagram for manufacturing a working wire and a reference wire, in accordance with some embodiments. The particular steps, combination of steps, and order of the steps for this process are provided for illustrative purposes only. Other processes with different steps, combinations of steps, or orders of steps can also be used to achieve the same or similar result. Features or functions described for one of the steps performed by one of the components may be enabled in a different step or component in some embodiments. Additionally, some steps may be performed before, after or overlapping other steps, in spite of the illustrated order of the steps. In addition, some of the functions (or alternatives for these functions) of the process are described elsewhere herein. For the working wire, the process starts by assigning an identifier such as a scannable code (e.g., bar code, quick response “QR” code, or the like) for tracking the progress during manufacturing. At block 205, an uncoated bare wire (e.g., raw material wire) which may be an electrical conducting wire comprised of, for example, platinum is used. At block 210, the wire is processed in a skiving and singulation station. Here, skiving and singulation are performed to shape the wire which may include removing portions of insulation from wire in a working area that will be used to take glucose measurements. Periodically, data sampling is performed after this process.

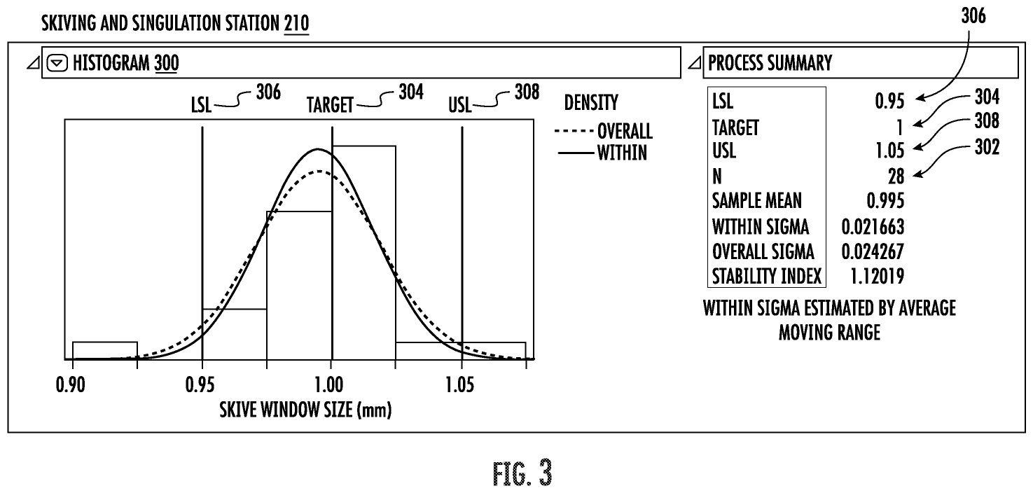

FIG. 3 depicts an example of data sampling after skiving and singulation during manufacturing of a sensor wire, in accordance with some embodiments. A histogram 300 shows data distribution of the uniformity of the created window from removing the portion of the insulation from the wire (skive window size). Removing the insulation in a uniform way enables consistent performance from sensor to sensor. In this data sampling, the data is collected for a sample size of 28 wires (labeled as 302) with a target skive window size 304 of 1.00 mm, noting a lower specification limit (LSL) 306 and an upper specification limit (USL) 308 based on a Six Sigma criteria.

Returning to FIG. 2, at block 215, ultrasonic and plasma processes may be applied to remove oxidation on the outer surfaces of the platinum wire to clean and prepare the surface of the wire for electropolymerization. Block 220 represents an electropolymerization (or electrodeposition) station. Electropolymerization is performed on the wire to form the interference membrane 121. This process provides a solid coating surrounding the wire which is conformal and very consistent having a controllable and repeatable layer formation. The interference membrane 121 is nonconducting of electrons but will pass ions and hydrogen peroxide at a preselected rate. Further, the interference membrane 121 may be formulated to be permselective for particular molecules. In one example, the interference membrane 121 is formulated and deposited in a way to restrict the passage of active molecules, which may act as contaminants to degrade the electrical conducting wire, or that may interfere with the electrical detection and transmission processes.

Advantageously, the interference membrane 121 precisely regulates the passage of hydrogen peroxide molecules to a wide surface area of the underlying wire. Further, formulation of the interference membrane 121 may be customized to allow for restricting or denying the passage of certain molecules to underlying layers, for example, restricting or denying the passage of large molecules or of particular target molecules.

Interference membrane 121 is a solid coating surrounding the wire. The interference membrane 121 may be precisely coated or deposited over the wire in a way that has a predictable and consistent passage of hydrogen peroxide. Further, the allowable area of interaction between the hydrogen peroxide and the surface of the wire is dramatically increased, as the interaction may occur anyplace along the skived portion of the wire. In this way, the interference membrane 121 enables an increased level of interaction between the hydrogen peroxide molecules in the surface of the wire such that the production of electrons is substantially amplified over prior art working electrodes. The interference membrane 121 enables the sensor to operate at a higher electron current, reducing the sensor's susceptibility to noise and interference from contaminants, and further enabling the use of less sophisticated and less precise electronics in the housing. In one example, the ability to operate at a higher electron flow allows electronics of the sensor to use standard operational amplifiers (op-amps), rather than the expensive precision op-amps required for prior art sensor systems. The resulting improved signal to noise ratio allows enable simplified filtering as well as streamlined calibration.

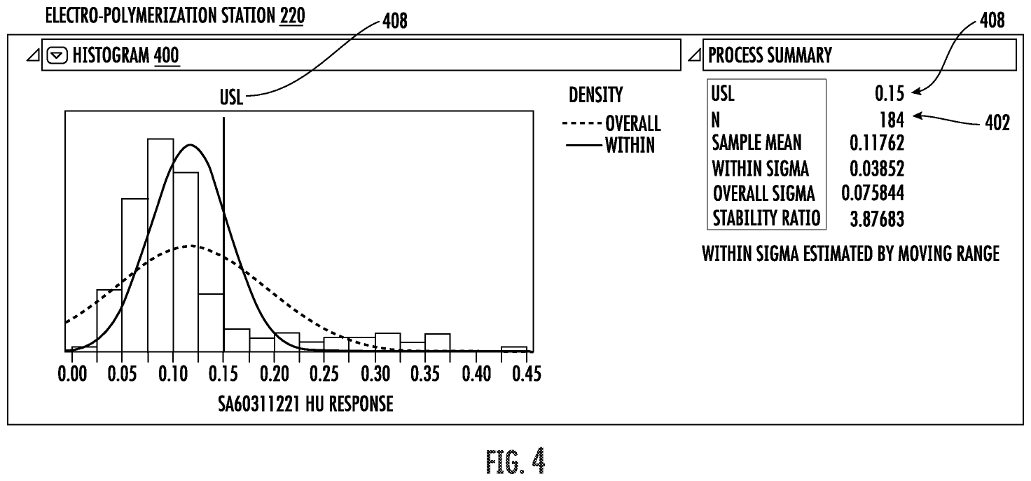

FIG. 4 depicts an example of data sampling after electropolymerization during manufacturing of a sensor wire, in accordance with some embodiments. A histogram 400 shows data distribution for a data sampling of 184 wires (labeled as 402) noting an upper specification limit (USL) 408 for HU (Hydroxyurea) Response according to a Six Sigma criteria. Hydroxyurea may be used as a known interferent that is a surrogate for interference performance.

In some embodiments, the enzyme membrane and the glucose limiting membrane are formed on the wire by a dipping process. Referring to FIG. 2, the aqueous dip station of block 225 forms the enzyme membrane 122 on the wire, and at block 230, the robotic dip station forms the glucose limiting membrane 123 on the wire, or in some embodiments, the robotic dipping station is used to also form the enzyme membrane 122. The dipping process is described in U.S. patent application Ser. No. 17/659,267, entitled “Coating a Working Wire for a Continuous Biological Sensor,” which is owned by the assignee of the present disclosure and incorporated herein by reference as if set forth in its entirety.

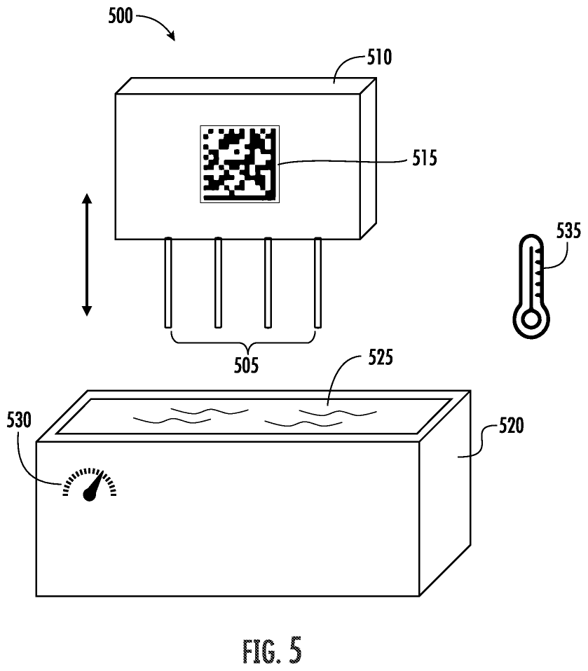

FIG. 5 is an isometric view of a dipping station 500 having a fixture 510 and a tub 520, in accordance with some embodiments. Multiple working wires 505 are mounted into the fixture 510. The fixture 510 is a holder, depicted as a block in this embodiment, for transporting the working wires 505 through a dipping process during manufacturing. The working wires 505 may be secured into the fixture 510 and mounted in a single row in this embodiment, spaced apart and extending from an edge of the fixture 510 so that each working wire 505 can be measured individually from various angles. In other embodiments the wires may be arranged in other ways such as in more than one row, aligned or staggered from each other, as long as sufficient space is between the wires to enable each wire to be measured separately. The fixture 510 may include an identifier such as a scannable code 515 (e.g., bar code, quick response “QR” code, or the like) for tracking the progress of the particular fixture 510 during manufacturing.

The tub 520 holds a dip solution 525 (or coating solution). The working wires 505 are submerged into the dip solution 525 to create the desired membrane on the wire. For example, the dipping process may be used to create the enzyme membrane 122 or glucose limiting membrane 123. Each membrane may require several dips (i.e., multiple coating iterations) to build up a desired thickness of the full membrane. Using several dipped layers to create a membrane can be advantageous in reducing the occurrence of pinholes in the membrane compared to creating the entire membrane thickness with a single dip.

The tub 520 may include one or more sensors 530 that monitor aspects of the dip solution such as viscosity or dip solution temperature. The system may also include environmental sensors 535 to monitor aspects of the ambient environment such as air temperature, relative humidity and airflow velocity. Embodiments of the present disclosure beneficially utilize these environmental sensors to provide input to a controller to adjust dipping parameters during manufacturing. In this manner, the controller automatically makes adjustments to account for process variations that are exceedingly challenging to manage manually. For example, changes in dip solution properties during the manufacturing process due to environmental factors can advantageously be compensated for in real-time. Lot-to-lot variations in dip solution viscosity or solids content can further affect how the environmental factors affect the dip solution. These impacts can also be accounted for by the present systems and methods.

Layer thicknesses formed in a dipping process are dependent upon several factors. Example dipping parameters and adjustments that may be made depending on the thickness measurements and other sensor information, in accordance with embodiments of the present disclosure, include the following:

Dip solution viscosity: A thicker dip solution provides a thicker layer per dip than a thinner dip solution. The dip solution has an initial known viscosity, which may change over time depending upon temperature, mixing, and evaporation.

Dip solution temperature: A cooler dip solution provides a thicker layer per dip than a warmer dip solution. The dip solution has an initial known temperature, which may change over time depending upon external temperature, mixing, and evaporation.

Immersion speed: Inserting the working wire into the dip solution at a slower rate will result in a thicker layer per dip than inserting the working wire at a faster rate. Based on all the expected parameters, an initial inserting speed is set, which may change for subsequent dips depending upon environmental conditions and actual thickness measurements.

Dwell time: Dwell time is the amount of time that the working wire remains fully immersed in the dip solution. A longer dwell time will result in a thicker layer per dip than a shorter dwell time. Based on all the expected parameters, an initial dwell time is set, which may change for subsequent dips depending upon environmental conditions and actual thickness measurements.

Withdrawal speed: Removing the working wire from the dip solution at a slower rate will result in a thinner layer per dip than a faster rate. Based on all the expected parameters, an initial withdrawal speed is set, which may change for subsequent dips depending upon environmental conditions and actual thickness measurements.

Airflow: Increasing airflow as the working wire comes out of dip solution lowers the solvent evaporation time and improves uniformity of the coating. Measurements of airflow velocity and/or relative humidity can be used to adjust dipping parameters.

Adjusting the parameters is automatically performed by specifically designed system/algorithms of U.S. patent application Ser. No. 17/659,267, thus improving manufacturing output by reducing defects and processing time. The parameters can also be adjusted in real-time, such as during the dipping process (e.g., between dips) in addition to between batches of dip solution. Dipping processes also tend to be non-linear in nature regarding the amount of coating that is deposited with each dip, which makes the thicknesses of the dipping layers difficult to predict. Interactions of the dipping parameters with other factors such as environmental conditions are also complex.

Using a wire plan as a customized program in the controller, the initial dip is performed and subsequent dips in a sequence of multiple dips are performed according to the wire plan along with applying adjustments made by controller. In embodiments, thicknesses of the dipped layers are measured as an in-line process—that is, as the working wires progress through the dipping process—and dipping parameters are adjusted as needed to achieve the required thicknesses within a target window of a thickness setpoint and/or within a predefined number of dips. For example, a total thickness of a membrane (e.g., enzyme membrane or glucose limiting membrane) may be desired to be 4 microns to 25 microns, such as 6 microns to 19 microns, where multiple coating layers are applied by the dipping process to form the total thickness. A target window for a desired setpoint thickness may be, for example, ±1 to ±3 microns, such as ±2 microns, of the setpoint thickness.

Conventional techniques typically use a fixed withdrawal speed and preset number of dips. After all the dips have been completed, the wires are measured and those that do not meet a diameter thickness specification, are rejected. In contrast, the present systems and methods measure the wires in-process; that is, after each dip. By using layer thickness measurements as feedback, dipping parameters can automatically be adjusted before the next dip is performed, to enable the working wire to be completed accurately, without impacting the processing time. For example, if the coating layers of the membrane are found to be thinner than expected, the dipping parameters can be adjusted to create thicker layers on the next dip so that the total thickness of the membrane can be met without adding more dips than originally planned. In another example, if the coating layers are found to be closer to the final desired thickness than expected, the dipping parameters can be adjusted to create a thinner layer on the next dip to avoid overshooting the diameter specification, which could result in a rejected part. Because interactions between dipping variables (e.g., environmental conditions, dip solution viscosity, immersion and withdrawal speeds, batch variations) are very complex in nature, and the tolerances of the layer thicknesses require extremely tight tolerances (e.g., within microns), the control and adjustments to achieve the accuracy needed are extremely difficult to perform manually or with conventional techniques. The systems and methods of the present disclosure provide control of layer dimensions and adjustment of dipping parameters that are unable to be achieved with conventional techniques.

For working wires that have already been dipped and cured, the automated measurement system measures the diameter of each wire using an in-line optical measurement tool (i.e., optical measurement tool used during the manufacturing process) to derive a coating thickness that has accumulated from the last dipping cycle. The optical measurement tool may be, for example, an optical micrometer that utilizes a laser beam to measure dimensions in a non-contact manner. The micrometer detects the size of the working wire by measuring the shadow of the object that is within the path of the laser beam. Because the robot is adjustable on multiple axes and can be very precisely controlled, each working wire in the fixture may have its thickness measured along its entire length and at different angles around its entire circumference. In this way, thickness is measured for every working wire at each dip for multiple lengthwise positions and angular rotations. In some embodiments, measurements can be made at more than one location along a length of a wire, and then the fixture can be rotated around a longitudinal axis of the wires so that the diameters are measured again along their length from a different orientation.

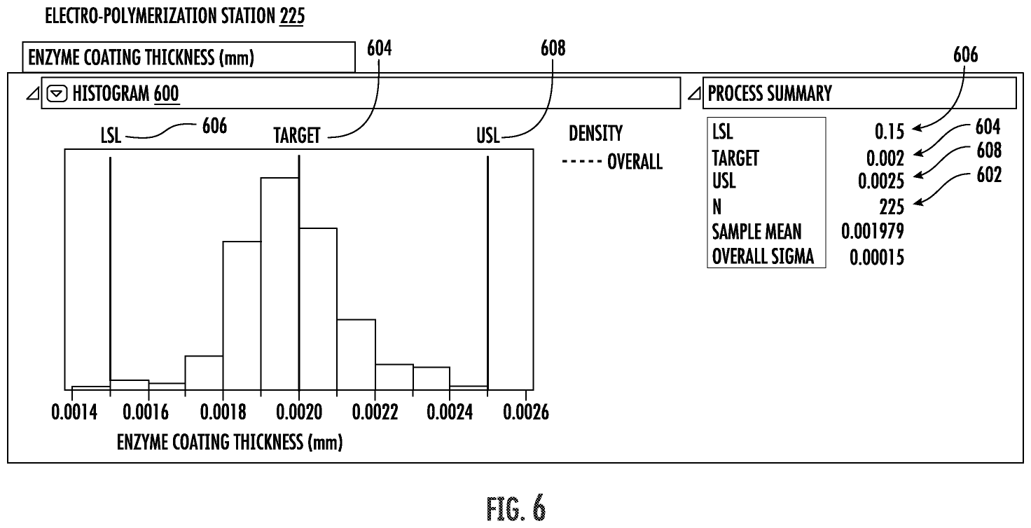

FIG. 6 depicts an example of data sampling after forming the enzyme membrane, in accordance with some embodiments. The data sampling is described with reference to FIG. 2, block 225. A histogram 600 shows the enzyme membrane coating thickness in which the target coating thickness 604 is 2.0 microns (μm). The data distribution is based on 225 wires (labeled as 602) noting a lower specification limit (LSL) 606 of 1.5 microns (μm) coating thickness and an upper specification limit (USL) 608 of 2.5 microns (μm) coating thickness for the enzyme membrane, according to a Six Sigma criteria. The yield of samples within the specification was 98%.

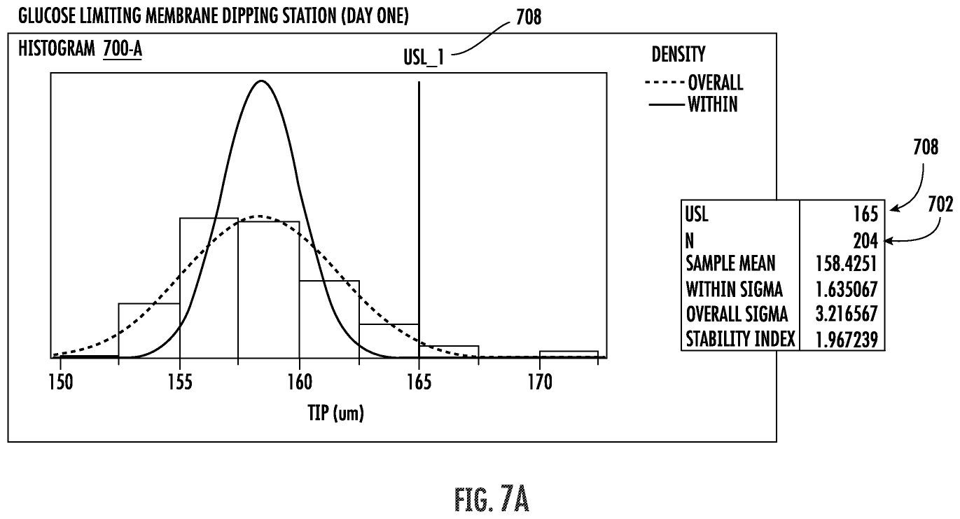

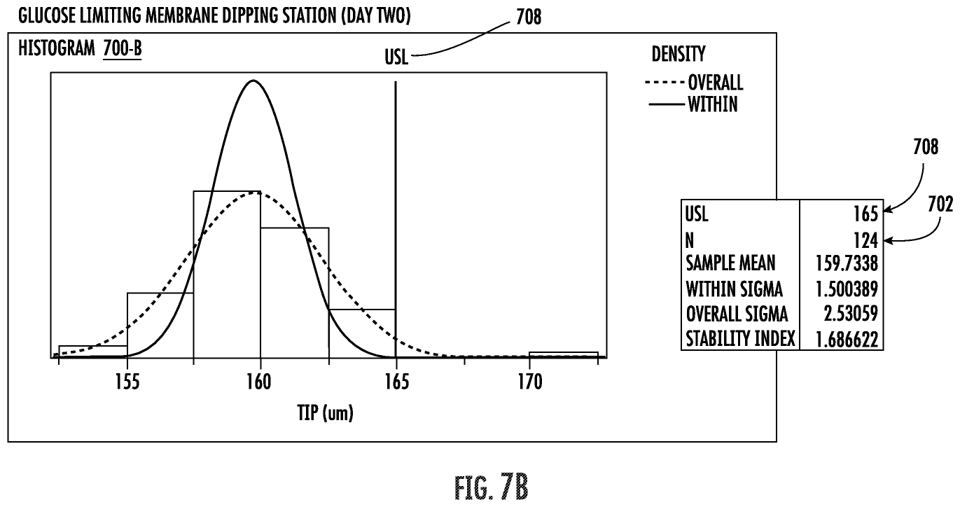

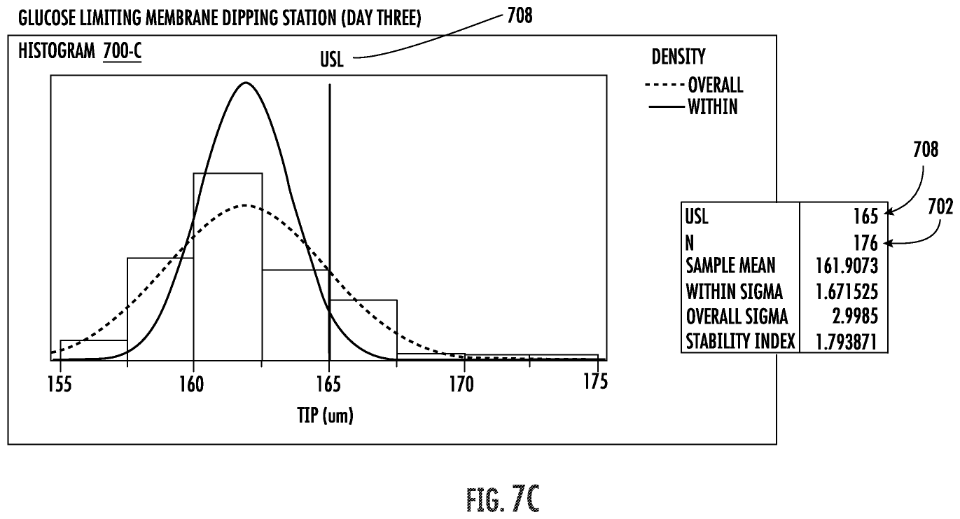

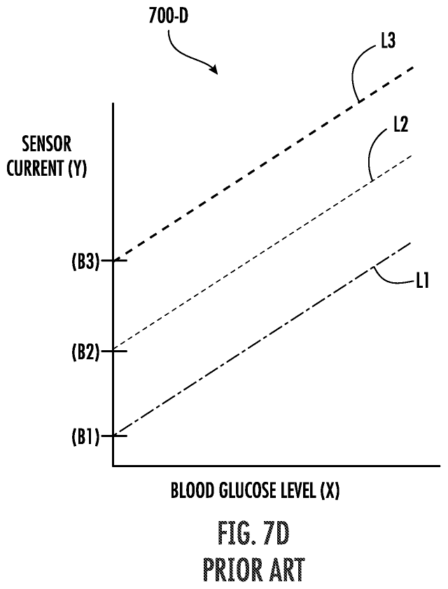

FIGS. 7A-7C depict examples of data samplings after forming the glucose limiting membrane on day one, day two and day three, in accordance with some embodiments. The data sampling is described with reference to FIG. 2, block 230. The sensor used in the data samplings are from three different manufacturing days which are not necessarily consecutive days. Each histogram, 700-A, 700-B and 700-C, show data distribution for the sample wires (number of samples labeled as 702) noting an upper specification limit (USL) for tip thickness 708 of 165 microns (μm) according to a Six Sigma criteria. The histograms 700-A, 700-B and 700-C in FIGS. 7A-7C respectively, demonstrate that the process is repeatable and similar on different days.

Referring to FIG. 2, blocks 250-265, the reference wire is also manufactured for the sensor. The reference wire may be comprised of silver and having a silver chloride layer surrounded by an ion limiting membrane that is nonconductive of electrons. The application of this ion limiting membrane over the silver/silver chloride layer desirably controls the current sensitivity of the sensor by controlling the flow of ions from the silver/silver chloride layer. In this way, current sensitivity may be advantageously controlled and defined. As will be understood, this can also act as a secondary method to control sensor sensitivity by controlling the chloride release from the electrode's surface.

The process for making the reference wire starts with assigning the wire an identifier or scannable code. The particular steps, combination of steps, and order of the steps for this process are provided for illustrative purposes only. Other processes with different steps, combinations of steps, or orders of steps can also be used to achieve the same or similar result. Features or functions described for one of the steps performed by one of the components may be enabled in a different step or component in some embodiments. Additionally, some steps may be performed before, after or overlapping other steps, in spite of the illustrated order of the steps. In addition, some of the functions (or alternatives for these functions) of the process have been described elsewhere herein. In some embodiments, the wire is comprised of silver. At block 255, the singulation station forms the wire. At block 260, a paste dip station applies a silver chloride layer to the silver wire by a dip coating process. At block 265, a robotic dip station applies the ion limiting membrane by a dip coating process.

After the completion of the working wire (blocks 205 through 230 of FIG. 2) and the reference wire (blocks 250 through 265 of FIG. 2), it will be understood that the association of the working wire with a reference wire to form the sensor may be accomplished in several ways. For example, the working wire and the reference wire may be placed side-by-side, formed concentrically, wrapped into a twisted relationship, layered, or formed into any other known physical relationships for a working wire and the associated reference wire.

Referring to FIG. 2, at block 240, a Cal Check Station performs calibration checks (i.e., “cal checks”) on the sensors. For example, linearity and factory sensitivity may be determined per sensor. The calibration check data may be obtained non-destructively and corresponds generally to the signal output of the sensor as a function of a plurality of input analyte concentrations. That is, in the calibration check test, a sensor is placed in test solutions and the corresponding output signal is measured.

FIG. 7D shows a sensitivity graph 700-D of the electrical response for a prior art CGM sensor. Graph 700-D has an X axis that represents the blood glucose level present in a user, which is typically measured in milligrams per deciliter (mg/dL). The Y-axis represents the amount of current flowing on the working wire (sensor current), which is typically measured in nanoamperes (nA). As illustrated in sensitivity graph 700-D, three user responses are shown by three different dashed lines L1, L2 and L3 which is the user response when the CGM sensor is implanted and actively in use. These user responses may be from three different users or may be from the same user at different times. As shown, although each of the user responses is linear, each has a very different baseline—labeled as B1, B2, B3 for lines L1, L2, L3, respectively. This baseline is the sensor current at a blood glucose level of zero and represents the amount of the sensor current that is attributed to noise or contaminant interference. This noise/contamination must be accounted for in a user-specific calibration process. The response for the sensor is generally linear and follows the algebraic equation of:

Y=AX+B (Equation 1),

where A (the slope of the line, rise over run) is the glucose sensitivity and B is the baseline. Generally, value “A” represents how sensitive the sensor is towards glucose and value “B” represents how specific the sensor is towards glucose. In some embodiments, a relationship between the factory sensitivity and a baseline is determined. The baseline is an electrical current generated by the sensor at a blood glucose level of zero. In some embodiments, the relationship is linear, and may be associated with the sensor. Prior art CGM sensors typically have a significantly high in-vivo baseline, which is caused by the in-vivo interferent compounds, such as acetaminophen, ascorbic acid and uric acid.

Embodiments herein enable the user response to be nearly the same in all cases due to the precise control of the dipping processes in making the sensors, and the user response crosses the X and Y axis at near-zero, which is referred to as the “intercept.” Accordingly, the sensor disclosed herein has a zero or near-zero intercept, and therefore does not need local user calibration, but can rely entirely upon factory calibration prior to shipment to the user. The generated electric current in response to an in-vivo glucose concentration of the patient may be, for example, less than 0.2 nA when the actual in-vivo glucose concentration in the patient is zero. Further, due to the consistent user response of the sensor disclosed herein, trustworthiness and accuracy in the resulting glucose reading is increased.

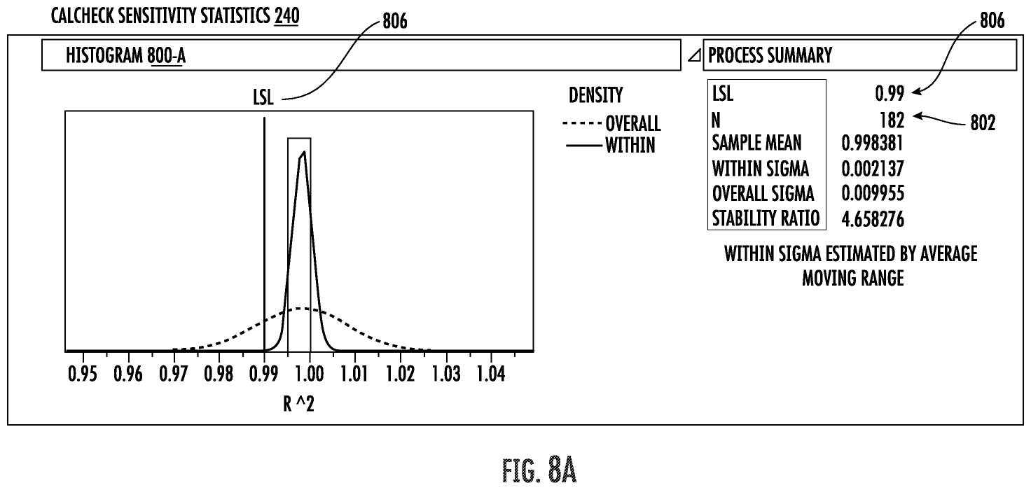

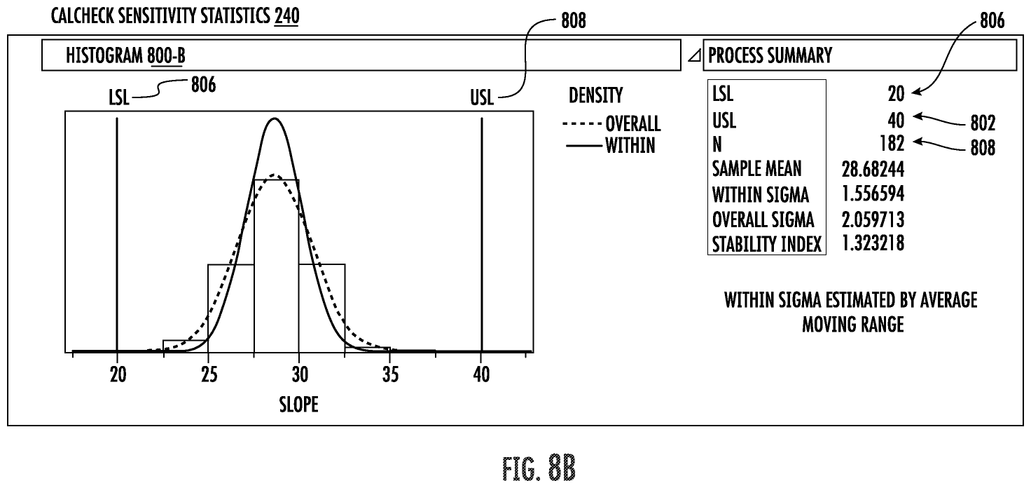

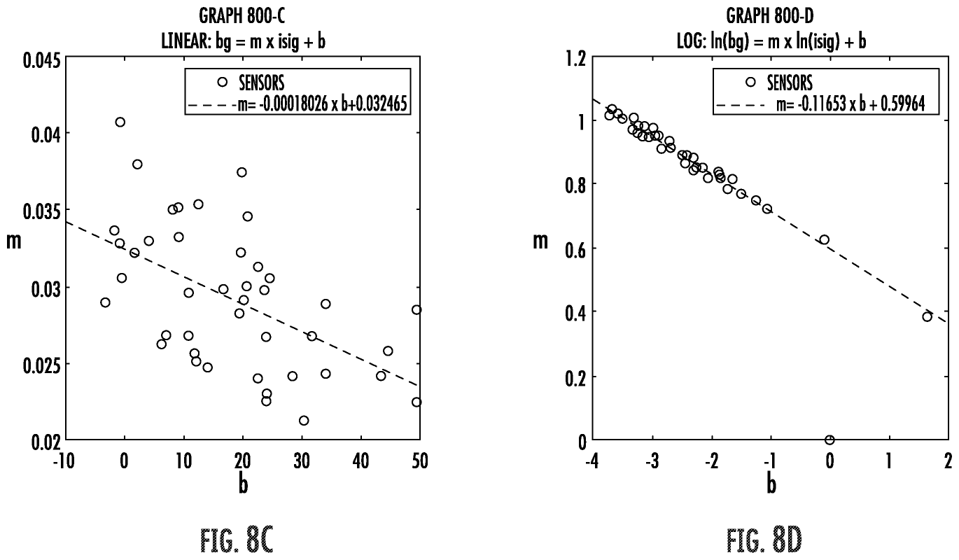

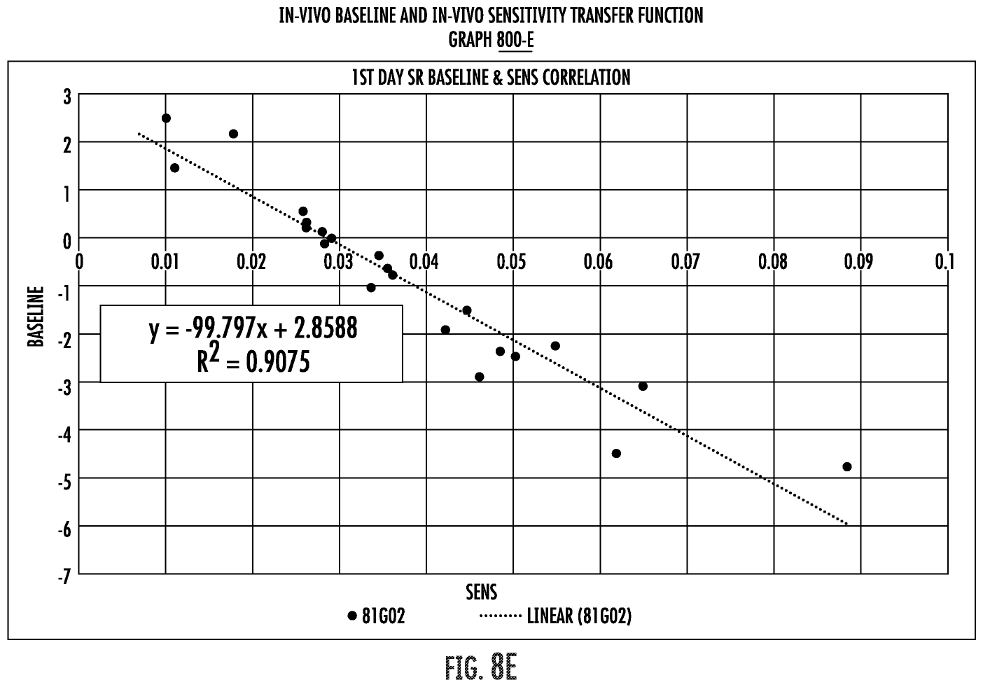

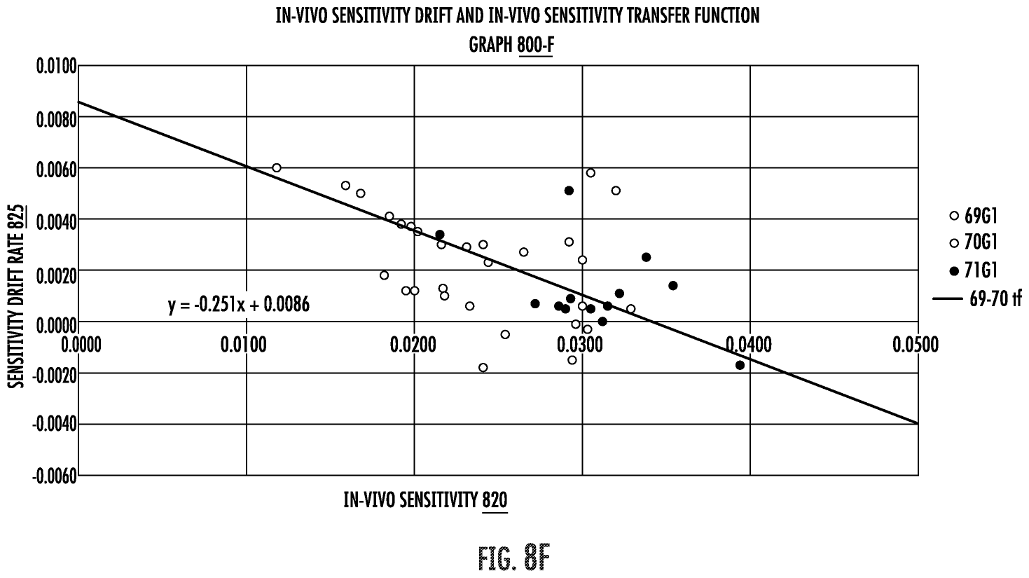

FIG. 8A depicts linearity statistics for sensors for a calibration check, in accordance with some embodiments. By strictly limiting the amount of glucose that can reach the enzyme membrane, linearity of the overall response is improved. A histogram 800-A according to Six Sigma shows that the methods and systems used herein achieve 0.99 in a consistent manner for the sample sensors (number of samples labeled as 802) noting a lower specification limit (LSL) 806. FIG. 8B depicts sensitivity statistics for sensors during a calibration check, in accordance with some embodiments. A histogram 800-B according to Six Sigma shows the sensitivity (e.g., slope) by measuring outputs as a function of analyte concentration and performing a linear regression. The data shown in the histogram 800-B is comprised of a sample size of sensors of 182 (labeled in FIG. 8B as 802) noting a lower specification limit (LSL) 806 and an upper specification limit (USL) 808.

Embodiments herein create a factory calibration by utilizing several factors of the sensor such as factory sensitivity of the sensor, enzyme membrane thickness, glucose limiting membrane thickness, overall sensor diameter, and drift profiles to develop a transfer function for a factory calibrated scheme. These factors are measured individually for every sensor to produce a 100% calibration check and 100% inspection for each wire/sensor during manufacture. The data can be monitored and tracked by the identifier or the scannable code for a particular sensor. For example, all of the data and parameters generated and gathered in the stations represented in blocks 205-265 of FIG. 2 can be associated with the identifier so that the data to manufacture each sensor is known. This can be saved and used in calculations such as the transfer function or algorithm. The data can also be used to predict in-vivo performance of the sensor. As such, each sensor is assigned its actual factory sensitivity, as it was measured at the factory, rather than being assigned a sensitivity based on population, lot or batch value. Conventionally in the art, sensors are calibrated in “lots” or “batches”. For example, a sensor is a member of a lot (or batch) of sensors in which sensors from the same and/or different manufacturing lots are assigned the same calibration factor despite variations due to manufacturing. The basis of assigning the calibration factor is only due to the sensor being a member of the same lot or batch. In some cases, this calibration factor may be associated with a sensor baseline sensitivity compared to an in-vivo sensitivity.

Through analysis, it has been determined in accordance with the present disclosure, that sensor sensitivity can be dependent on sensor (e.g., working wire) membrane thicknesses but other subtle differences in sensors may also influence sensitivity. For example, two sensors may have the same sensitivity but have different membrane thicknesses (e.g., enzyme membrane and/or glucose limiting membrane). In another example, two sensors may have the same sensitivity but have different working wire diameters. In each example, despite the sensors having the same sensitivity, they perform differently from one another such as in response time or glucose reading output. Accordingly, embodiments herein determine a calibration scheme based not only on sensitivity but also differences due to manufacturing variations such as in the working wire diameter where the working wire diameter includes the enzyme membrane thickness and the glucose limiting membrane thickness.

In embodiments herein, the enzyme membrane thickness, glucose limiting membrane thickness, and overall sensor diameter are measured per sensor based on dipping parameters during manufacturing. This data may be used in the calibration which is assigned to the particular sensor that the data is based upon. In some embodiments, only the enzyme membrane thickness is used in the calibration, only the glucose limiting membrane thickness is used in the calibration, or the working wire diameter including the enzyme membrane thickness and the glucose limiting membrane thickness is used in the calibration. In prior art systems, the thickness of each membrane cannot be measured or determined due to the conventional processes used to form the membranes.

As described herein, the membrane thicknesses require extremely tight tolerances (e.g., within microns). There is a correlation between membrane thickness (e.g., the enzyme membrane thickness and/or glucose limiting membrane) and the performance in-vivo such as sensor sensitivity and sensitivity drift. Bench testing (e.g., performing calibration checks) of the manufactured sensors may be used to collect data such as sensitivity of the sensor. Since the sensor is tracked and monitored with a scannable code, all the associated data to manufacture the particular sensor is known and available. The data includes the membrane thicknesses, working wire diameter, sensor diameter, and dipping parameters used during the dip coating process for each membrane. During bench testing, the sensitivity of the sensor may be recorded for the first few hours then the data can be used to create a transfer function which may be a polynomial, linear, logarithmic, or exponential, or other type of function. Ideally, this is as close to a 1:1 sensitivity. From this, a sensitivity drift profile and an algorithm is generated.

Since the thickness of the enzyme membrane and the glucose limiting membrane are measured and associated with the sensor, the data can be used to determine correlations or relationships. For example, there may be a correlation between enzyme membrane thickness and longevity or sensitivity changes over time. This may be used in to refine an algorithm for calibration instead of using only sensitivity as the variable.

Through data collection performed in relation to the present disclosure, drift profiles for sensor samples show sensitivity responses in-vitro for sensors having the same sensitivity but different measured membrane thicknesses. For example, for a sensor having a membrane thickness within specification and near a lower specification limit (e.g., a thinner membrane thickness), the sensor performance drifts upward quickly over time such as beginning on day 2 and continuing for consecutive days. In another example, for a sensor having a membrane thickness within specification and near a mid-specification limit, the sensor performance drift is flat over time such as until day 10 and then tails off. In another example, for a sensor having a membrane thickness within specification and near an upper specification limit (e.g., a thicker membrane thickness), the sensor performance drifts down over time in a continuous, linear fashion. Again, in these examples, all the sensors have the same sensitivity. Rather than only considering sensitivity, other factors such as membrane thickness, first dipping parameters for one membrane and second dipping parameters for another membrane, may be used in the factory calibration. Put another way, the membrane thicknesses have a range that is considered within the specification. By tracking the working wire diameter and/or sensor diameter (including membrane thicknesses), the sensitivity drift or performance can be predicted based on the actual membrane thicknesses.

A drift profile can be automatically generated for each manufactured sensor based a correlation between the parameters used in the dip coating of the enzyme membrane and the parameters used in the dip coating of the glucose limiting membrane, to the working wire diameter and sensitivity. In some embodiments, the greater the slope of the sensor (e.g., sensitivity of the sensor), the greater the rate of drift of the sensor. A plurality of drift profiles can be embedded in the algorithm and stored in a computing platform, such as a cloud server. The algorithm may include other data such as prior knowledge developed in clinical studies or historical data, and data generated for the sensor during manufacture such as the bench data, membrane thicknesses, dipping parameters, and the like. The platform stores all the data and the algorithm. The CGM system includes the sensor which cooperates with electronics in a small housing typically worn on the skin. The CGM system is described in U.S. Pat. No. 11,471,081, entitled “Continuous Glucose Monitoring Device,” which is owned by the assignee of the present disclosure and incorporated herein by reference as if set forth in its entirety. The electronics in the CGM system includes a microprocessor and a transmitter. The transmitter is configured to transmit data through a communication technology, such as a WiFi system, Bluetooth® wireless technology, Bluetooth® Low Energy, cellular communications, satellite communications or the like, as well as combinations thereof. A device such as a smartphone, computer, router, hub, cellular network transceiver or the like or combinations thereof receives the data.

The algorithm may be a series of calculations and in some embodiments, there is one algorithm assigned to all sensors. In other embodiments, there may be a plurality of algorithms such as three to six algorithms, using the same data set. One of the algorithms may be assigned to a sensor based on some of the data collected during the manufacturing process. For example, the enzyme membrane thickness and the sensitivity may be predictive of which algorithms to use for the sensor to obtain the best performance. Since a lot of data is collected during the manufacturing process, there are more potential factors to consider for correlation to predict the change in performance of the sensor over time (e.g., drift). In contrast, it is known in the art that data such as membrane thicknesses and dipping parameters are not collected, and only sensitivity is determined for a batch of sensors, not each sensor. In the present embodiments, data is collected during the manufacturing process per sensor and all data is carried forward with each sensor by its identifier. This data is a potential use for determining an algorithm or a series of algorithms. For example, there may be six algorithms using the same data set in a prospective fashion. One of the six algorithms is assigned to a sensor based on heuristics rules such as when the data for the sensor is A+B, algorithm one is assigned, and when the data for the sensor is A+B+C, algorithm two is assigned. A, B, C may be data collected during the manufacturing process such as enzyme membrane thickness, glucose limiting membrane thickness, dipping parameters, baseline (sensor current at a blood glucose level of zero and represents the amount of the sensor current that is attributed to noise or contaminant interference), or mathematical relationships or correlations derived from the data. This helps improve the overall performance and prediction in-vivo of the sensor.

The algorithm for sensitivity of the sensor changing over time may be developed as a time-based system such as using time to create a series of functions which may be polynomial, logarithmic, or any mathematical model. For example, for a time-based system, there may be three linear regions over time such as 15 days. The first region may be day one to day two of in-vivo use, the second region may be day two to day ten of in-vivo use, and the third region may be day ten to day 15 of in-vivo use. A linear equation is associated with each region which is time-based and predicts sensitivity of the sensor. A nominal seed value (e.g., baseline) may be the calibration check value on day zero normalized to one. As one specific example, the equations or linear functions may be 0.88x for the first region, 0.01x+1 for the second region, and −0.015x+1 for the third region where x is time. In this way, a drift profile is created by the performance being divided into time regions and each time region predicts the sensitivity based on a mathematical model. In some embodiments, the mathematical model may be derived from historical data from the enzyme membrane thicknesses and the glucose limiting membrane thicknesses. In some embodiments, the mathematical model may be derived from the data historical data, bench data, in-vivo studies and field data from the membrane thicknesses, dipping parameters, and the like. In another example, a different algorithm may be used based on heuristics such as electrical current ranges. In some embodiments, the algorithm may be a glycemic range-based system as opposed to the time-based system considering hypoglycemic and hyperglycemic conditions. For example, in a hypoglycemic condition which is the lowest current, the seed value—baseline—may not be zeroed out but instead be used in the calculation.

In conventional sensor systems, algorithms for sensor calibration are developed using fixed values for sensor baseline, sensor sensitivity, and sensor drift rate. The fixed values are commonly a midpoint of a range. By doing so, the sensor needs to be manufactured according to narrow specifications in order for the sensor to fit within the algorithm. In contrast, in some embodiments of the present disclosure, the algorithm is developed from the sensor baseline, sensor sensitivity, and sensor drift rate which are each developed from different transfer functions. This enables a more accurate algorithm when compared to using a fixed parameter approach. It also enables the sensor to be manufactured having a wider range of specifications accommodating different ranges of the baseline, sensitivity, and drift while fitting into the algorithm, thus increasing the yield of sensors within specification in the manufacture process.

FIG. 8C is a graph 800-C of baseline of the sensor and sensitivity of the sensor, in accordance with some embodiments. There may be a relationship between baseline current of the sensor and in-vivo current that is not glucose related such as:

bg=m x isig+b (Equation 2),

where as:

m=−0.00018026 x b+0.032465 (Equation 3),

Isig is current, b is the baseline, bg is background where background refers to any in-vivo current that is not glucose related, and m is the slope. Graph 800-C shows that there is not a clear correlation between the quantities. For example, the data points do not have a close fit to the proposed linear correlation.

FIG. 8D is a graph 800-D of the log of baseline of the sensor and the log of sensitivity of the sensor from the data in FIG. 8C, in accordance with some embodiments. It was unexpectedly found that when the log of the baseline of the sensor and the log of the sensitivity of the sensor is plotted on a graph, there is a predictable linear relationship between current and background which may be used in sensor algorithms (e.g., sensor calibration) to predict behavior of the sensor. For example, in the field, the baseline of the sensor is known from the sensor's manufactured history (e.g., layer thicknesses). The current isig may be measured in the field, and then the correlation may be used to predict how much of the current is due to background noise instead of glucose. In one example, the relationship may be:

In(bg)=m x In(isig)+b (Equation 4),

where as:

m=−0.11653 x b+0.59964 (Equation 5).

This may be used in the generation of the factory calibration of the sensor and be impactful for “edge cases” such as hypoglycemia or for the early days of use of the sensor such as day one to day three. For example, the processor may determine a baseline of the sensor based on the enzyme membrane thickness and the glucose limiting membrane thickness. The baseline is an electrical current generated by the sensor at a blood glucose level of zero. The processor may determine a correlation between the baseline and the sensitivity of the sensor. In some embodiments, the correlation may be a log-log relationship. The processor may use the correlation to determine a background current of the sensor. The background current is a current that is not glucose related.

FIG. 8E is a graph of in-vivo baseline and in-vivo sensitivity, in accordance with some embodiments. For baseline and sensitivity of the sensor, a linear transfer function is found. The graph shows the sensitivity of sensors on the x-axis and the baseline of the sensor on the y-axis, and the correlation may be linear such as:

y=−99.797x+2.8588 (Equation 6),

and R²=0.9075, indicating a close fit of the data points with this linear correlation. The linear correlation may be used as a transfer function to predict how the baseline changes as a function of sensitivity. FIG. 8F is a graph 800-F of in-vivo sensitivity drift and in-vivo sensitivity, in accordance with some embodiments. For drift and sensitivity of the sensor, a linear transfer function is generated. Graph 800-F shows examples of the sensitivity of sensor 820 on the x-axis and the sensitivity drift rate (e.g., drift profile) of the sensor 825 on the y-axis. The correlation may be linear such as:

y=−0.251x+0.0086 (Equation 7),

which can be used as a transfer function to predict how the sensitivity drifts over time, for different values of sensitivity. For example, historical data of various test samples of in-vivo sensitivity drift may be used, and a relationship may be determined between the historical data of in-vivo sensitivity of a plurality of sensors and the drift profile of the sensor. In some embodiments, the relationship may be linear, and the relationship may be associated with the sensor. The relationship may be part of the algorithm to predict the sensitivity of sensor over time. For example, during use of the sensor by the patient, the sensor may output a glucose reading based on the relationship. The correlations and transfer functions of FIGS. 8C-8F were unique insights that were recognized in relation to the present disclosure, which were enabled because of the detailed membrane thickness measurements and 100% tracking of individual sensor data.

In some embodiments, a processor of the platform is configured to communicate with the CGM system and may receive a confirmation from the transmitter in the CGM system that the sensor in the CGM system is in active use by a patient. The processor may identify the drift profile, relationships, correlations, and all the relevant data (e.g., membrane thicknesses) for the sensor associated with the identifier of the sensor. From this, the processor can execute an appropriate algorithm to compensate for the sensitivity drift of the sensor based on the drift profile. As a result, the blood glucose reading or output of the sensor may be based on, for example, the drift profile, or other data associated with the sensor. This occurs automatically and without user input enabling highly accurate performance of the sensor. By using this approach of the processor communicating with the sensor and applying the algorithm to automatically adjust the output of the sensor, it eliminates the need for the user to recalibrate the sensor by using the finger prick method and obtaining blood glucose readings. In embodiments, the platform and CGM system also connect and communicate with an electronic device such as a mobile phone or computing tablet through an app.

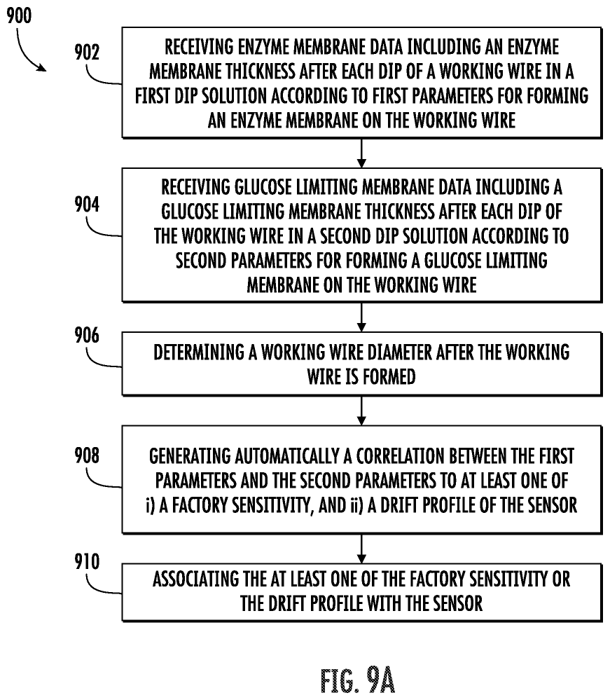

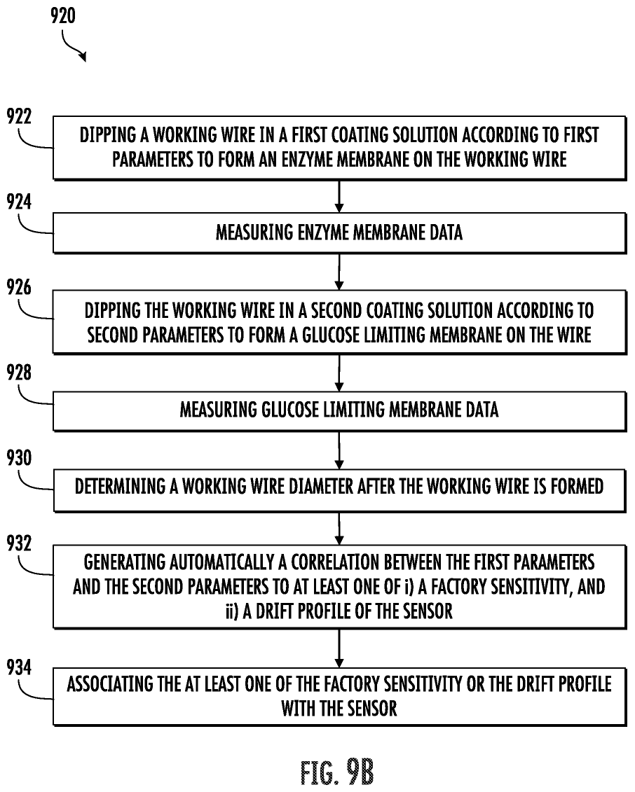

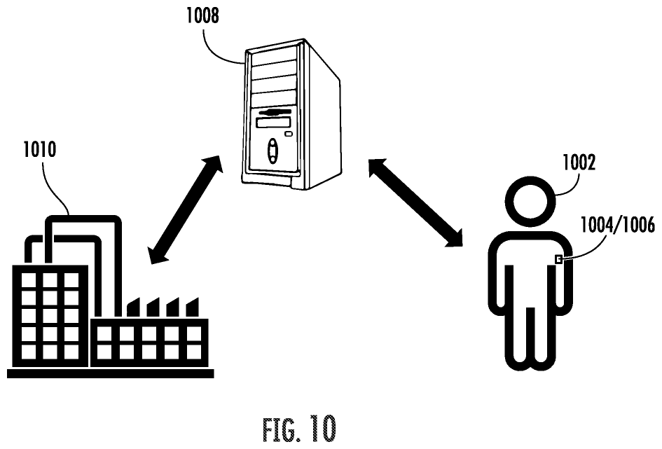

FIG. 9A is a flowchart for a method 900 for a factory calibration of a sensor of a continuous glucose monitoring (CGM) system, in accordance with some embodiments. The particular steps, combination of steps, and order of the steps for this process are provided for illustrative purposes only. Other processes with different steps, combinations of steps, or orders of steps can also be used to achieve the same or similar result. Features or functions described for one of the steps performed by one of the components may be enabled in a different step or component in some embodiments. Additionally, some steps may be performed before, after or overlapping other steps, in spite of the illustrated order of the steps. In addition, some of the functions (or alternatives for these functions) of the process have been described elsewhere herein.

At block 902, a processor receives enzyme membrane data including an enzyme membrane thickness after each dip of a working wire in a first dip solution according to first parameters for forming an enzyme membrane on the working wire. At block 904, the processor receives glucose limiting membrane data including a glucose limiting membrane thickness after each dip of the working wire in a second dip solution according to second parameters for forming a glucose limiting membrane on the working wire. At block 906, the processor determines a working wire diameter after the working wire is formed. The working wire diameter includes the enzyme membrane thickness and the glucose limiting membrane thickness. The formed working wire includes an interference membrane, the enzyme membrane and the glucose limiting membrane. At block 908, the processor in communication with the CGM system, automatically generates a correlation between the first parameters and the second parameters to at least one of a factory sensitivity and a drift profile of the sensor. The drift profile predicts a sensitivity of the sensor over time. At block 910, the processor associates the at least one of the factory sensitivity or the drift profile with the sensor. The sensor outputs a glucose reading during in-vivo use based on the at least one of the factory sensitivity or the drift profile.

FIG. 9B is a flowchart for a method 920 for a factory calibration of a sensor of a continuous glucose monitoring (CGM) system, in accordance with some embodiments. The particular steps, combination of steps, and order of the steps for this process are provided for illustrative purposes only. Other processes with different steps, combinations of steps, or orders of steps can also be used to achieve the same or similar result. Features or functions described for one of the steps performed by one of the components may be enabled in a different step or component in some embodiments. Additionally, some steps may be performed before, after or overlapping other steps, in spite of the illustrated order of the steps. In addition, some of the functions (or alternatives for these functions) of the process have been described elsewhere herein.

At block 922, a working wire is dipped in a first coating solution according to first parameters to form an enzyme membrane on the working wire. At block 924, enzyme membrane data including an enzyme membrane thickness is measured. At block 926, the working wire is dipped in a second coating solution according to second parameters to form a glucose limiting membrane on the wire. At block 928, glucose limiting membrane data including a glucose limiting membrane thickness is measured. At block 930, a processor determines a working wire diameter after the working wire is formed. The working wire diameter includes the enzyme membrane thickness and the glucose limiting membrane thickness. The formed working wire includes an interference membrane, the enzyme membrane and the glucose limiting membrane. At block 932, the processor in communication with the CGM system, automatically generates a correlation between the first parameters and the second parameters to at least one of a factory sensitivity, and a drift profile of the sensor. The drift profile predicts a sensitivity of the sensor over time. At block 934, the processor associates the at least one of the factory sensitivity or the drift profile with the sensor. The sensor outputs a glucose reading during in-vivo use based on the at least one of the factory sensitivity or the drift profile.

In a non-limiting example, it is known that the average sensitivity of the sensor is 25 picoamperes/mg/dL. When the sensor sensitivity is 40 picoamperes/mg/dL, the minimal current may be 1,000 picoamperes, and when the sensor sensitivity measures 400, the maximum current may be 10,000 picoamperes. During the first day of sensor use by a patient, monitoring the current may show that the rolling current average is 80% of the maximum for a sustained period of time. From this, it may be determined that the sensor is a high sensitivity sensor, and a high sensitivity projection for the drift profile is automatically assigned to the sensor without user input.

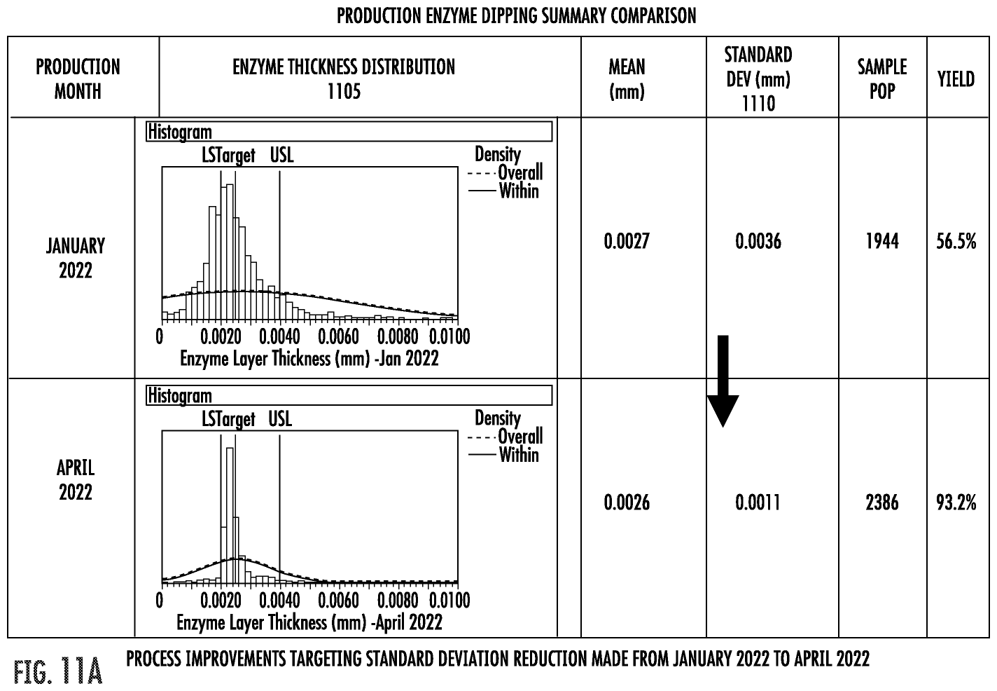

FIG. 10 is a schematic of a CGM system in-use by a patient, in accordance with some embodiments. A patient 1002 (e.g., user) may subcutaneously insert the sensor 1004 of the CGM system in their body for use. The electronics 1006 of the CGM system communicate with a computing platform 1008, such as a cloud server. The computing platform 1008 communicates with the manufacturing facility 1010. In this way, the manufacturing facility 1010 provides data and information to the computing platform 1008 such as data and information for the sensitivity values and drift profiles. The drift profiles may be generated by the computing platform 1008 or by the manufacturing facility 1010 and are stored in the computing platform 1008. The computing platform 1008 communicates with the electronics 1006 of the CGM system to provide the drift profiles to compensate for sensitivity of the sensor 1004. The communication may be through a communication technology, such as a WiFi system, Bluetooth® wireless technology, Bluetooth® Low Energy, cellular communications, satellite communications or the like, as well as combinations thereof.

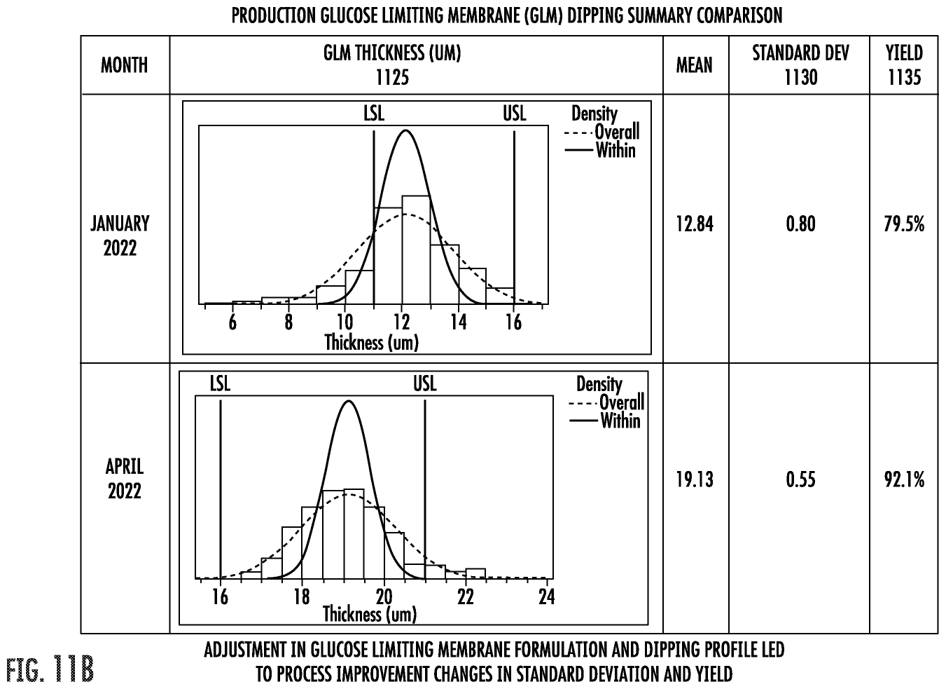

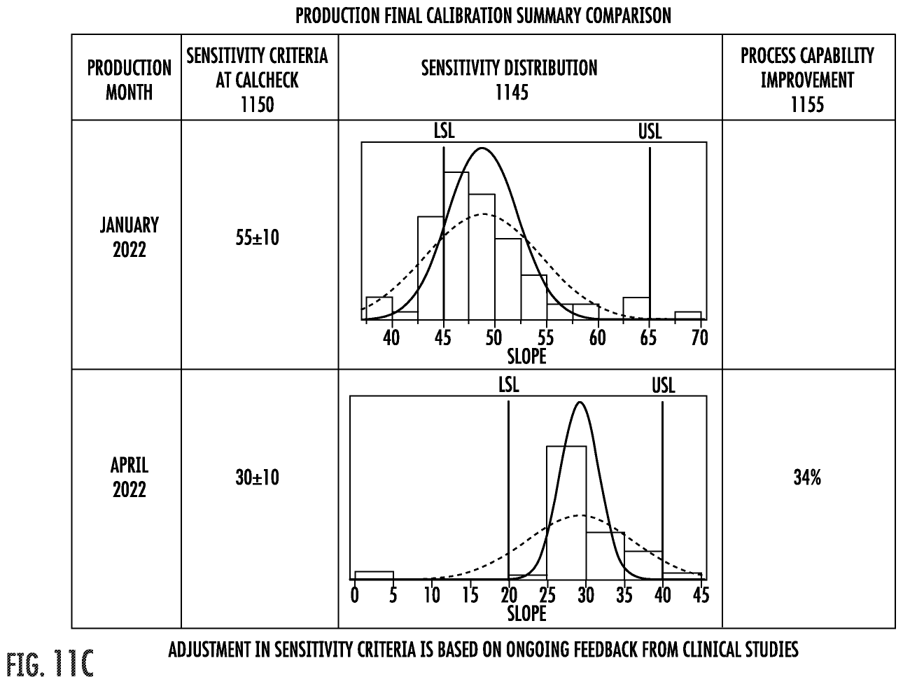

FIGS. 11A-11C are tables detailing process improvements, in accordance with some embodiments. FIG. 11A shows process improvements targeting standard deviation reduction that were acheived over a three-month period. By improving the enzyme thickness distribution 1105, the standard deviation 1110 in millimeters improved from 0.0036 to 0.0011. FIG. 11B shows process improvements to the glucose limiting membrane thickness 1125 by adjusting the glucose limiting membrane formulation and dipping profile. The improvements are noted by the standard deviation 1130 reduction from 0.80 to 0.55. Also, the yield 1135 increased from 79.5% to 92.1%. FIG. 11C shows a production calibration summary comparison. During the calibration check, the sensitivity distribution 1145 dramatically improved as noted in the sensitivity criteria 1150. The sensitivity criteria 1150 at calibration check changed from 55±10 in January to 30±10 in April resulting in a 34% improvement in process capability 1155.

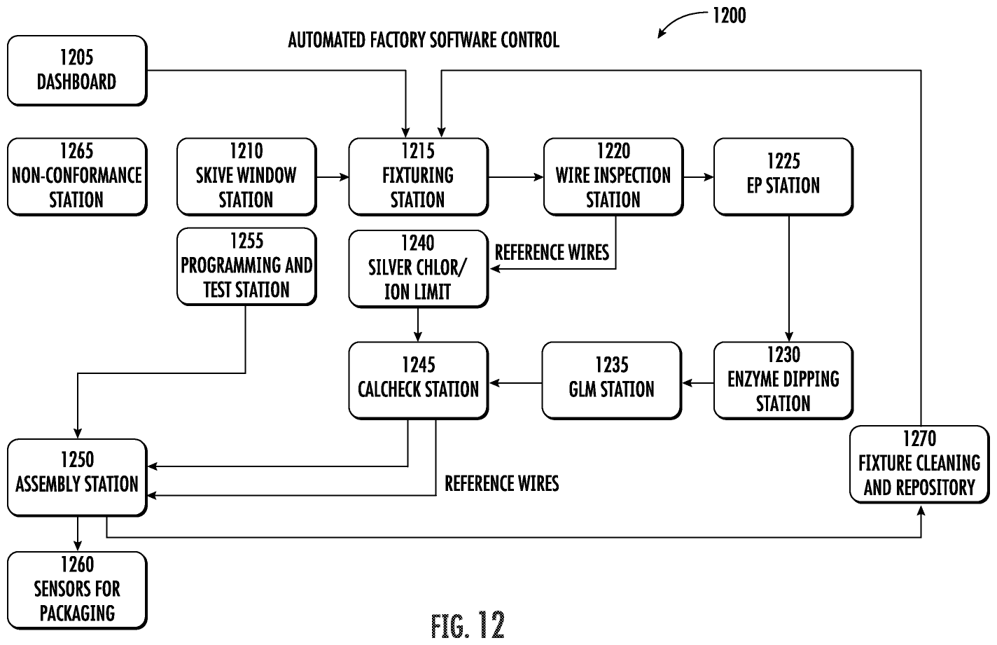

FIG. 12 illustrates an automated factory software control process, in accordance with some embodiments. The particular steps, combination of steps, and order of the steps for this process are provided for illustrative purposes only. Other processes with different steps, combinations of steps, or orders of steps can also be used to achieve the same or similar result. Features or functions described for one of the steps performed by one of the components may be enabled in a different step or component in some embodiments. Additionally, some steps may be performed before, after or overlapping other steps, in spite of the illustrated order of the steps. In addition, some of the functions (or alternatives for these functions) of the process have been described elsewhere herein. A process 1200 starts at block 1205 with the dashboard of the process. The dashboard may be implemented on a device and is a graphical user interface (GUI) that provides a visual representation of information, data, metrics, and the like. The dashboard monitors and manages the various aspects of the system and method as described herein. At block 1210, the wires (e.g., the working wire and the reference wire of the sensor) begin in the skiving and singulation station as described with reference to FIG. 2, blocks 210 and 215. Data may be displayed when the wire is positioned in a fixture at block 1215, and for the inspection at block 1220. To form the working wire, the wire proceeds to block 1225, and the software monitors the parameters as the working wire enters the electropolymerization station to form the interference membrane as described with reference to FIG. 2, block 220. At blocks 1230 and 1235, the software is used to control the formation of the enzyme membrane and the glucose limiting membrane as described in FIG. 2, blocks 225 and 230 respectively.

To form the reference wire, after the inspection station of block 1220, the reference wire, in some embodiments, a third party may have applied the silver/silver chloride coating at a specified thickness (block 260 of FIG. 2). The wire then proceeds to block 1240 where the ion limiting membrane is formed by the dip coating process (as described in block 265 of FIG. 2). The working wire and reference wire proceed to block 1245 where calibration checks are performed on the wires and further data may be recorded. At block 1250, the working wire and reference wire are assembled to form the sensor and as described in block 1245, calibration checks are performed on the sensor and further data may be collected. At block 1255, the sensors are programmed, and testing may be executed. At block 1260, the sensors are packaged for sale. During calibration checks and throughout the process, the wires or sensors may be flagged as out of specifications. These sensors are quarantined and sent to block 1265, the non-conformance station. At this station, the sensors may be scraped or repaired and returned to the process if possible. At block 270, the fixtures may be cleaned, and all the data is stored in a repository.

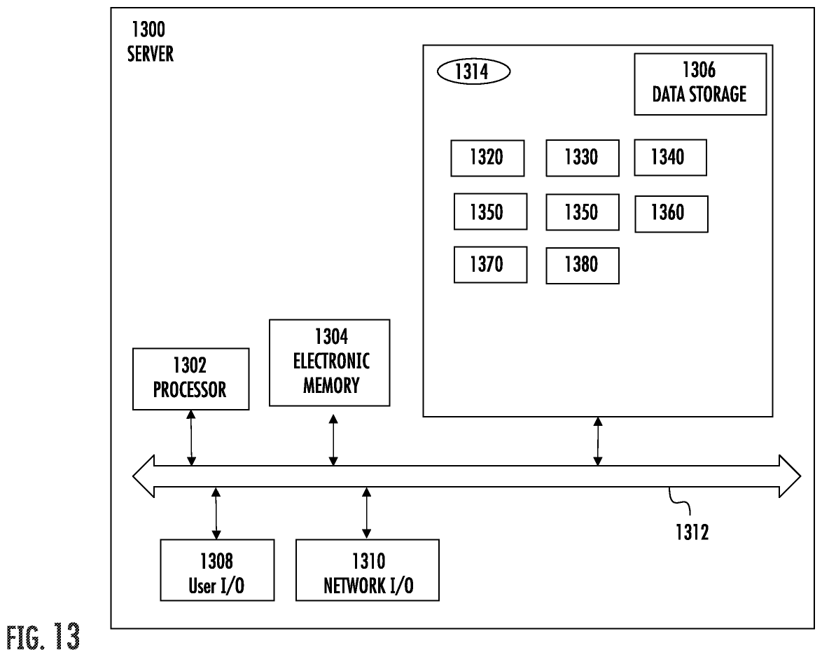

FIG. 13 is a simplified schematic diagram showing an example computer system 1300 (representing any combination of one or more of the computer systems) for use in the computing platform 1008 for executing any of the programs described herein, in accordance with some embodiments. Other embodiments may use other components and combinations of components. For example, the computer system 1300 may represent one or more physical computer devices or servers, such as web servers, rack-mounted computers, network storage devices, desktop computers, laptop/notebook computers, etc., depending on the complexity of the computing platform 1008. In some embodiments implemented at least partially in a cloud network potentially with data synchronized across multiple geolocations, the server 1300 may be referred to as one or more cloud servers. In some embodiments, the functions of the server 1300 are enabled in a single computer device. In more complex implementations, some of the functions of the computing system are distributed across multiple computer devices, whether within a single server farm facility or multiple physical locations. In some embodiments, the server 1300 functions as a single virtual machine.

In some embodiments wherein the server 1300 represents multiple computer devices, some of the functions of the server 1300 are implemented in some of the computer devices, while other functions are implemented in other computer devices. In the illustrated embodiment, the server 1300 generally includes at least one processor 1302, a main electronic memory 1304, a data storage 1306, a user I/O 1308, and a network I/O 1310, among other components not shown for simplicity, connected or coupled together by a data communication subsystem 1312.

The processor 1302 represents one or more central processing units on one or more PCBs (printed circuit boards) in one or more housings or enclosures. In some embodiments, the processor 1302 represents multiple microprocessor units in multiple computer devices at multiple physical locations interconnected by one or more data channels. When executing computer-executable instructions for performing the above described functions of the server 1300 in cooperation with the main electronic memory 1304, the processor 1302 becomes a special purpose computer for performing the functions of the instructions.

The main electronic memory 1304 represents one or more RAM modules on one or more PCBs in one or more housings or enclosures. In some embodiments, the main electronic memory 1304 represents multiple memory module units in multiple computer devices at multiple physical locations. In operation with the processor 1302, the main electronic memory 1304 stores the computer-executable instructions executed by, and data processed or generated by, the processor 1302 to perform the above described functions of the server 1300.

The data storage 1306 represents or comprises any appropriate number or combination of internal or external physical mass storage devices, such as hard drives, optical drives, network-attached storage (NAS) devices, flash drives, etc. In some embodiments, the data storage 1306 represents multiple mass storage devices in multiple computer devices at multiple physical locations. The data storage 1306 generally provides persistent storage (e.g., in a non-transitory computer-readable media or machine-readable medium 1314) for the programs (e.g., computer-executable instructions) and data used in operation of the processor 1302 and the main electronic memory 1304.

In some embodiments, the main electronic memory 1304 and the data storage 1306 include all, or a portion of the programs and data (e.g., represented by 1320-1380) required by the processor 1302 to perform the methods, processes and functions disclosed herein (e.g., in FIGS. 1-12). Under control of these programs and using this data, the processor 1302, in cooperation with the main electronic memory 1304, performs the above-described functions for the computing platform 1008.

The user I/O 1308 represents one or more appropriate user interface devices, such as keyboards, pointing devices, displays, etc. In some embodiments, the user I/O 1308 represents multiple user interface devices for multiple computer devices at multiple physical locations. A system administrator, for example, may use these devices to access, setup and control the server 1300.

The network I/O 1310 represents any appropriate networking devices, such as network adapters, etc. for communicating through the computing platform 1008. In some embodiments, the network I/O 1310 represents multiple such networking devices for multiple computer devices at multiple physical locations for communicating through multiple data channels.

The data communication subsystem 1312 represents any appropriate communication hardware for connecting the other components in a single unit or in a distributed manner on one or more PCBs, within one or more housings or enclosures, within one or more rack assemblies, within one or more geographical locations, etc.

The server 1300 includes a memory storing executable instructions (loaded from the data storage 1306) and the processor 1302. The processor 1302 is coupled to the memory 1304 and performs the methods by executing the instructions stored in the memory 1304. The non-transitory computer-readable media 1314 includes instructions that, when executed by the processor 1302, cause the processor 1302 to perform operations including the methods 900 and 920 as described herein.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A method for factory calibration of a sensor of a continuous glucose monitoring (CGM) system comprising:
receiving, by a processor, enzyme membrane data including an enzyme membrane thickness after each dip of a working wire in a first dip solution according to first parameters for forming an enzyme membrane on the working wire;
receiving, by the processor, glucose limiting membrane data including a glucose limiting membrane thickness after each dip of the working wire in a second dip solution according to second parameters for forming a glucose limiting membrane on the working wire;
determining, by the processor, a working wire diameter after the working wire is formed, wherein the working wire diameter includes the enzyme membrane thickness and the glucose limiting membrane thickness, and wherein the formed working wire includes an interference membrane, the enzyme membrane and the glucose limiting membrane;
generating automatically, by the processor in communication with the CGM system, a correlation between the first parameters and the second parameters to at least one of i) a factory sensitivity, and ii) a drift profile of the sensor, wherein the drift profile predicts a sensitivity of the sensor over time; and
associating, by the processor, the at least one of the factory sensitivity or the drift profile with the sensor, wherein the sensor outputs a glucose reading during in-vivo use based on the at least one of the factory sensitivity or the drift profile.

2. The method of claim 1, wherein the drift profile is divided into time regions and each time region predicts the sensitivity based on a mathematical model.

3. The method of claim 2, wherein the mathematical model is derived from historical data from enzyme membrane thicknesses and glucose limiting membrane thicknesses.

4. The method of claim 1, further comprising:
determining, by the processor, a relationship between the factory sensitivity and a baseline, wherein the baseline is an electrical current generated by the sensor at a blood glucose level of zero; and
associating, by the processor, the relationship with the sensor.

5. The method of claim 4, wherein the relationship is linear.

6. The method of claim 1, further comprising:
determining, by the processor, a relationship between historical data of in-vivo sensitivity of a plurality of sensors and the drift profile of the sensor; and
associating, by the processor, the relationship with the sensor.

7. The method of claim 6, wherein the relationship is linear.

8. The method of claim 1, further comprising:
determining, by the processor, a baseline of the sensor based on the enzyme membrane thickness and the glucose limiting membrane thickness, wherein the baseline is an electrical current generated by the sensor at a blood glucose level of zero;
determining, by the processor, a correlation between the baseline and the sensitivity of the sensor, the correlation being a log-log relationship; and
using, by the processor, the correlation to determine a background current of the sensor, wherein the background current is a current that is not glucose related.

9. The method of claim 1, further comprising:
receiving, by the processor from a transmitter in the CGM system, a confirmation that the sensor of the CGM system is in use by a patient;
identifying, by the processor, the drift profile of the sensor; and
communicating, by the processor to a microprocessor of the CGM system, a new sensitivity for the sensor based on the drift profile of the sensor, wherein the sensor outputs a glucose reading based on drift profile.

10. The method of claim 1, wherein the interference membrane is formed by an electropolymerization process.

11. The method of claim 1, further comprising measuring interference membrane data including an interference membrane thickness after forming the interference membrane on the working wire.

12. The method of claim 1, wherein the first parameters and the second parameters include at least one of a dip solution viscosity, dip solution temperature, immersion speed, dwell time, withdrawal speed, and airflow.

13. The method of claim 1, wherein each dip of the working wire comprises:
dipping the working wire into a first dip solution according to the first parameters for the forming of the enzyme membrane or dipping the working wire into a second dip solution according to the second parameters for the forming of the glucose limiting membrane;
measuring, as an in-line process, a plurality of diameters along a length of the working wire using an automated measurement system;
determining, by the processor in communication with the automated measurement system, a thickness difference, the thickness difference being a difference between a thickness setpoint and an aggregate criteria for the plurality of diameters; and
calculating, by the processor, adjusted parameters for the dipping process based on the thickness difference.

14. A method for factory calibration of a sensor of a continuous glucose monitoring (CGM) system comprising:
dipping a working wire in a first coating solution according to first parameters to form an enzyme membrane on the working wire;
measuring enzyme membrane data, wherein the enzyme membrane data includes an enzyme membrane thickness;
dipping the working wire in a second coating solution according to second parameters to form a glucose limiting membrane on the working wire;
measuring glucose limiting membrane data, wherein the glucose limiting membrane data includes a glucose limiting membrane thickness;
determining, by a processor, a working wire diameter after the working wire is formed, wherein the working wire diameter includes the enzyme membrane thickness and the glucose limiting membrane thickness, and wherein the formed working wire includes an interference membrane, the enzyme membrane and the glucose limiting membrane;
generating automatically, by the processor in communication with the CGM system, a correlation between the first parameters and the second parameters to at least one of i) a factory sensitivity, and ii) a drift profile of the sensor, wherein the drift profile predicts a sensitivity of the sensor over time; and
associating, by the processor, the at least one of the factory sensitivity or the drift profile with the sensor, wherein the sensor outputs a glucose reading during in-vivo use based on the at least one of the factory sensitivity or the drift profile.

15. The method of claim 14, wherein the drift profile is divided into time regions and each time region predicts the sensitivity based on a mathematical model.

16. The method of claim 15, wherein the mathematical model is derived from historical data from enzyme membrane thicknesses and glucose limiting membrane thicknesses.

17. The method of claim 14, further comprising:
determining, by the processor, a relationship between the factory sensitivity and a baseline, wherein the baseline is an electrical current generated by the sensor at a blood glucose level of zero; and
associating, by the processor, the relationship with the sensor.

18. The method of claim 14, further comprising:
determining, by the processor, a relationship between historical data of in-vivo sensitivity of a plurality of sensors and the drift profile of the sensor; and
associating, by the processor, the relationship with the sensor.

19. The method of claim 14, further comprising:
determining, by the processor, a baseline of the sensor based on the enzyme membrane thickness and the glucose limiting membrane thickness, wherein the baseline is an electrical current generated by the sensor at a blood glucose level of zero;
determining, by the processor, a correlation between the baseline and the sensitivity of the sensor, the correlation being a log-log relationship; and
using, by the processor, the correlation to determine a background current of the sensor, wherein the background current is a current that is not glucose related.

20. The method of claim 14, further comprising:
receiving, by the processor from a transmitter in the CGM system, a confirmation that the sensor of the CGM system is in use by a patient;
identifying, by the processor, the drift profile of the sensor; and
communicating, by the processor to a microprocessor of the CGM system, a new sensitivity for the sensor based on the drift profile of the sensor, wherein the sensor outputs a glucose reading based on drift profile.
